(19) United States
(12) Patent Application Publication
(10) Pub. No.: US 2008/0274157 A1
(43) Pub. Date: Nov. 6, 2008

(54) Cartilage implant plug with fibrin glue and method for implantation

(76) Inventors: Gordana Vunjak-Novakovic, New York, NY (US); Katherine G. Truncale, Hillsborough, NJ (US); Moon Hae Sunwoo, Old Tappan, NY (US); Arthur A. Gertzman, Lebanon, NJ (US)

Correspondence Address: GREENBERG TRAURIG, LLP, 200 PARK AVE., P.O. BOX 677, FLORHAM PARK, NJ, 07932, US

(21) Appl. No.: 12/079,629

(22) Filed: Mar. 26, 2008

Related U.S. Application Data
(62) Division of application No. 10/960,960, filed on Oct. 12, 2004, now abandoned.
Application No. 10/960,960 is a continuation-in-part of application No. 10/815,778, filed on Apr. 2, 2004.
Application No. 10/815,778 is a continuation-in-part of application No. 10/424,765, filed on Apr. 29, 2003, now Pat. No. 7,067,123.

(51) Int. Cl. A61K 35/32; A61F 2/00; A61P 19/00

(52) U.S. Cl. 424/423; 424/549

(57) ABSTRACT

The invention is directed toward a cartilage repair assembly comprising a shaped structure of subchondral bone with an integral overlying cartilage cap which is treated to remove cellular debris and proteoglycans and milled cartilage in a bioabsorbable carrier. The shaped structure is dimensioned to fit in a drilled bore in a cartilage defect area so that said shaped bone and cartilage cap when centered in the bore does not engage the side wall of the bore and is positioned from the side wall of the bone a distance ranging from 10 microns to 1000 microns and is surrounded by milled cartilage and a fibrin thrombin glue. A method for inserting the assembly into a cartilage defect area is disclosed.

### RELATED APPLICATIONS

This is a divisional application of U.S. patent application Ser. No. 10/960,960 filed Oct. 12, 2004, which is a continuation-in-part application of U.S. patent application Ser. No. 10/815,778 filed Apr. 2, 2004 and U.S. patent application Ser. No. 10/424,765 filed Apr. 29, 2003, now U.S. Pat. No. 7,067,123. All of the foregoing applications are incorporated by reference herein in their entirety.

### FIELD OF INVENTION

The present invention is generally directed toward a surgical implant and is more specifically directed toward an implant for a joint having a cartilage face and bone body for implantation in a shoulder, hip, elbow, ankle, knee or temporomandibular joint.

### BACKGROUND OF THE INVENTION

Articular cartilage injury and degeneration present medical problems to the general population which are constantly addressed by the orthopedic surgeon. Every year in the United States, over 500,000 arthroplastic or joint repair procedures are performed. These include approximately 125,000 total hip and 150,000 total knee arthroplastics and over 41,000 open and arthroscopic procedures to repair cartilaginous defects of the knee. Chen et al. “Repair of Articular Cartilage Defects: Part 1, Basic Science of Cartilage Healing”, American Journal of Orthopaedics 1999, January: 31-33.

In the knee joint, the articular cartilage tissue forms a lining which faces the joint cavity on one side and is linked to the subchondral bone plate by a narrow layer of calcified cartilage tissue on the other. Articular cartilage (hyaline cartilage) consists primarily of extracellular matrix with a sparse population of chondrocytes distributed throughout the tissue. Articular cartilage is composed of chondrocytes, type II collagen fibril network, proteoglycans and water. Active chondrocytes are unique in that they have a relatively low turnover rate and are sparsely distributed within the surrounding matrix. The collagens give the tissue its form and tensile strength and the interaction of proteoglycans with water give the tissue its stiffness for compression, resilience and durability. The hyaline cartilage provides a low friction bearing surface over the bony parts of the joint. If the lining becomes worn or damaged resulting in lesions, joint movement may be painful or severely restricted. Whereas damaged bone typically can regenerate successfully, hyaline cartilage regeneration is quite limited.

Articular cartilage lesions generally do not heal, or heal only partially under certain biological conditions due to the lack of nerves, blood vessels and a lymphatic system. The limited reparative capabilities of hyaline cartilage generally result in the generation of repair tissue that lacks the structure and biomechanical properties of normal cartilage. Generally, the healing of the defect results in a fibrocartilaginous repair tissue that lacks the structure and biomechanical properties of hyaline cartilage and degrades over the course of time. Articular cartilage lesions are frequently associated with disability and with symptoms such as joint pain, locking phenomena and reduced or distributed function. These lesions are difficult to treat because of the distinctive structure and function of hyaline cartilage. Such lesions are believed to progress to severe forms of osteoarthritis. Osteoarthritis is the leading cause of disability and impairment in middle-aged and older individuals, entailing significant economic, social and psychological costs. Each year, osteoarthritis accounts for as many as 39 million physician visits and more than 500,000 hospitalizations. By the year 2020, arthritis is expected to affect almost 60 million persons in the United States and to limit the activity of 11.6 million persons. Jackson et al., “Cartilage Substitutes, Overview of Basic Science and Treatment Options”, Journal of American Academy of Orthopedic Surgeons, 2001, 9:37-52.

There are many current therapeutic methods being used. None of these therapies has resulted in the successful regeneration of durable hyaline-like tissue that withstands normal joint loading and activity over prolonged periods. Currently, the techniques most widely utilized clinically for cartilage defects and degeneration are not articular cartilage substitution procedures, but rather lavage, arthroscopic debridement, and repair stimulation. The direct transplantation of cells or tissue into a defect and the replacement of the defect with biologic or synthetic substitutions presently accounts for only a small percentage of surgical interventions. The optimum surgical goal is to replace the defects with cartilage-like substitutes so as to provide pain relief, reduce effusions and inflammation, restore function, reduce disability and postpone or alleviate the need for prosthetic replacement.

Lavage and arthroscopic debridement involve irrigation of the joint with solutions of sodium chloride, Ringer or Ringer and lactate. The temporary pain relief is believed to result from removing degenerative cartilage debris, proteolytic enzymes and inflammatory mediators. These techniques provide temporary pain relief, but have little or no potential for further healing.

Repair stimulation is conducted by means of drilling, abrasion arthroplasty or microfracture. Penetration into the subchondral bone opens access of the hosts bone marrow derived stem cells and induces bleeding and fibrin clot formation which promotes initial repair, however, the tissue formed is fibrous in nature and not durable. Pain relief is temporary as the tissue exhibits degeneration, loss of resilience, stiffness and wear characteristics over time.

The peritoneum and perichondrium have been shown to contain mesenchymal progenitor cells capable of differentiation and proliferation. They have been used as grafts in both animal and human models to repair articular defects. Few patients over 40 years of age have obtained good clinical results, which most likely reflects the decreasing population of osteochondral progenitor cells with increasing age. There have also been problems with fixation and stability of the grafts, which result in their displacement or loss from the repair site.

Transplantation of cells grown in culture provides another method of introducing a new cell population into chondral and osteochondral defects. Carticel® is a commercial process to culture the patient's own cartilage cells for use in the repair of cartilage defects in the knee joint and is marketed by Genzyme Biosurgery in the United States and Europe. The procedure uses arthroscopy to take a biopsy from a healthy, less loaded area of knee articular cartilage. Enzymatic digestion of the harvested tissue releases the cells that are sent to a laboratory where they are grown for a period ranging from 2-5 weeks to achieve a 10-fold increase in cell mass. Once cultivated, the autologous cells are injected during a more open and extensive knee procedure into areas of defective cartilage where it is hoped that they will facilitate the repair of damaged tissue. An autologous periosteal flap with cambium layer facing down is used to seal the transplanted cells in place and act as a mechanical barrier. Fibrin glue is used to seal the edges of the flap. Proponents of this procedure report that it produces satisfactory results, including the ability to return to demanding physical activities, in more than 80% of patients and that biopsy specimens of the tissue in the graft sites show hyaline-like cartilage repair. However, long term studies of this procedure in rabbits and dogs showed limited success and showed degradation at the implant site. The original study report has been criticized for not being a prospective controlled randomized study and for lack of quantitative or mechanical. Of interest, a 14 year follow-up of a similar patient group that underwent diagnostic arthroscopy in combination with one of several treatments (removal of bone bodies, shaving, Pride drilling) had good to excellent knee function in 78% of the patients. Thus, further studies are needed to assess the function and durability of the new tissue to determine whether it improves joint function and delays or prevents joint degeneration.

As with the perichondrial graft, patient/donor age may compromise the success of this procedure as chondrocyte population decreases with increasing age. Disadvantages to this procedure include the need for two separate surgical procedures, potential damage to surrounding cartilage when the periosteal patch is sutured in place, the requirement of demanding complex microsurgical techniques, and the expensive cost of the procedure which is currently not covered by insurance.

Osteochondral transplantation or mosaicplasty involves excising all injured or unstable tissue from the articular defect and creating cylindrical holes in the base of the defect and underlying bone. These holes are filled with autologous cylindrical plugs of healthy cartilage and bone in a mosaic fashion. The osteochondral plugs are harvested from a lower weight-bearing area of lesser importance in the same joint. This technique, shown in Prior Art FIG. 2, can be performed as arthroscopic or open procedures. Reports of results of osteochondral plug autografts in a small numbers of patients indicate that they decrease pain and improve joint function. Factors that can compromise the results include donor site morbidity, effects of joint incongruity on the opposing surface of the donor site, damage to the chondrocytes at the articular margins of the donor and recipient sites during preparation and implantation, and collapse or settling of the graft over time. The limited availability of sites for harvest of osteochondral autografts restricts the use of this approach to treatment of relatively small articular defects and the healing of the chondral portion of the autograft to the adjacent articular cartilage remains a concern.

Transplantation of large allografts of bone and overlying articular cartilage is another treatment option that involves a greater area than is suitable for autologous cylindrical plugs, as well as for a non-contained defect. The advantages of osteochondral allografts are the potential to restore the anatomic contour of the joint, lack of morbidity related to graft harvesting, greater availability than autografts and the ability to prepare allografts in any size to reconstruct large defects. Clinical experience with fresh and frozen osteochondral allografts shows that these grafts can decrease joint pain, and that the osseous part of an allograft can heal to the host bone and the chondral part can function as an articular surface. Drawbacks associated with this methodology in the clinical situation include the scarcity of fresh donor material and problems connected with the handling and storage of frozen tissue. Fresh allografts carry the risk of immune response or disease transmission. Musculoskeletal Transplant Foundation (MTF) has preserved fresh allografts in a media that maintains a cell viability of 50% for 35 days at 4° C.

A number of United States patents have been specifically directed towards bone plugs which are implanted into a bone defect. Examples of such bone plugs are U.S. Pat. No. 4,950,296 issued Aug. 21, 1990 which discloses a bone graft device comprising a cortical shell having a selected outer shape and a cavity formed therein for receiving a cancellous plug, and a cancellous plug fitted into the cavity in a manner to expose at least one surface; U.S. Pat. No. 6,039,762 issued Mar. 21, 2000 having a cylindrical shell with an interior body of deactivated bone material and U.S. Pat. No. 6,398,811 issued Jun. 4, 2002 directed to a bone spacer which has a cylindrical cortical bone plug with an internal throughgoing bore designed to hold a reinforcing member. U.S. Pat. No. 6,383,221 issued May 7, 2002 discloses an invertebral implant having a substantially cylindrical body with a throughgoing bore dimensioned to receive bone growth materials.

U.S. Pat. No. 6,379,385 issued Apr. 30, 2002 discloses an implant base body of spongious bone material into which a load carrying support element is embedded. The support element can take the shape of a diagonal cross or a plurality of cylindrical pins. See also, U.S. Pat. No. 6,294,187 issued Sep. 25, 2001 which is directed to a load bearing osteoimplant made of compressed bone particles in the form of a cylinder. The cylinder is provided with a plurality of throughgoing bores to promote blood flow through the osteoimplant or to hold a demineralized bone and glycerol paste mixture. U.S. Pat. No. 6,096,081 issued Aug. 1, 2000 shows a bone dowel with a cortical end cap or caps at both ends, a brittle cancerous body and a throughgoing bore.

A number of patents in the prior art show the use of bone putty, pastes or gels to fill bone defects. U.S. Pat. No. 5,290,558 issued Mar. 1, 1994 discloses a flowable demineralized bone powder composition using an osteogenic bone powder with large particle size ranging from about 0.1 to about 1.2 cm. mixed with a low molecular weight polyhydroxy compound possessing from 2 to about 18 carbons including a number of classes of different compounds such as monosaccharides, disaccharides, water dispersible oligosaccharides and polysaccharides.

Another such bone gel is disclosed in U.S. Pat. No. 5,073,373 issued Dec. 17, 1991. Bone lamellae in the shape of threads or filaments retaining low molecular weight glycerol carrier are disclosed in U.S. Pat. No. 5,314,476 issued May 24, 1994 and U.S. Pat. No. 5,507,813 issued Apr. 16, 1996 and the tissue forms described in these patents are known commercially as the GRAFTON® Putty and Flex, respectively.

U.S. Pat. No. 5,356,629 issued Oct. 18, 1994 discloses making a rigid gel in the nature of a bone cement to fill defects in bone by mixing biocompatible particles, preferably polymethylmethacrylate coated with polyhydroxyethylmethacrylate in a matrix selected from a group which lists hyaluronic acid to obtain a molded semi-solid mass which can be suitably worked for implantation into bone. The hyaluronic acid can also be utilized in monomeric form or in polymeric form preferably having a molecular weight not greater than about one million Daltons. It is noted that the nonbioabsorbable material which can be used to form the biocompatible particles can be derived from xenograft bone, homologous bone, autogenous bone as well as other materials. The bioactive substance can also be an osteogenic agent such as demineralized bone powder, moralized cancellous bone, aspirated bone marrow and other autogenous bone sources. The average size of the particles employed is preferably about 0.1 to about 3.0 mm, more preferably about 0.2 to about 1.5 mm, and most preferably about 0.3 to about 1.0 mm. It is inferentially mentioned but not taught that particles having average sizes of about 7,000 to 8,000 microns, or even as small as about 100 to 700 microns can be used.

U.S. Pat. No. 4,172,128 issued Oct. 23, 1979 discloses a demineralized bone material mixed with a carrier to reconstruct tooth or bone material by adding a mucopolysaccharide to a mineralized bone colloidal material. The composition is formed from a demineralized coarsely ground bone material, which may be derived from human bones and teeth, dissolved in a solvent forming a colloidal solution to which is added a physiologically inert polyhydroxy compound such as mucopolysaccharide or polyphonic acid in an amount which causes orientation when hydrogen ions or polyvalent metal ions are added to form a gel. Example 25 of the '128 patent notes that mucopolysaccharides produce pronounced ionotropic effects and that hyaluronic acid is particularly responsible for spatial cross-linking.

U.S. Pat. No. 6,030,635 issued Feb. 29, 2000 and U.S. Pat. No. 6,437,018 issued Aug. 20, 2002 are directed toward a malleable bone putty and a flowable gel composition for application to a bone defect site to promote new bone growth at the site which comprises a new bone growth inducing compound of demineralized lyophilized allograft bone powder. The bone powder has a particle size ranging from about 100 to about 850 microns and is mixed in a high molecular weight hydrogel carrier which contains a sodium phosphate saline buffer.

The use of implants for cartilage defects is much more limited. Aside from the fresh allograft implants and autologous implants, U.S. Pat. No. 6,110,209 issued Nov. 5, 1998 shows the use of an autologous articular cartilage cancellous bone paste to fill arthritic defects. The surgical technique is arthroscopic and includes debriding (shaving away loose or fragmented articular cartilage), followed by moralizing the base of the arthritic defect with an awl until bleeding occurs. An osteochondral graft is then harvested from the inner rim of the intercondylar notch using a trephine. The graft is then moralized in a bone graft crusher, mixing the articular cartilage with the cancellous bone. The paste is then pushed into the defect and secured by the adhesive properties of the bleeding bone. The paste can also be mixed with a cartilage stimulating factor, a plurality of cells, or a biological glue. All patients are kept non-weight bearing for four weeks and used a continuous passive motion machine for six hours each night. Histologic appearance of the biopsies have mainly shown a mixture of fibrocartilage with hyaline cartilage. Concerns associated with this method are harvest site morbidity and availability, similar to the mosaicplasty method.

U.S. Pat. No. 6,379,367 issued Apr. 30, 2002 discloses a plug with a base membrane, a control plug, and a top membrane which overlies the surface of the cartilage covering the defective area of the joint.

U.S. Pat. No. 6,488,033 issued Dec. 3, 2002 discloses an allograft plug with a cartilage cap which is surface contour matched to the surface of a condyle defect area which is to be replaced. The allograft plug is transplanted in an interference fit within the cavity site which remains after a condylar defect is removed from a patient's condyle.

### SUMMARY OF THE INVENTION

A cartilage allograft construct assembly comprising a plug with a bone base and cartilage cap for treating articular cartilage defects. The plug is used together with a milled cartilage glue which surrounds the plug in a bore which has been cut into the patient to remove the lesion area. The process for inserting the construct plug is to arthroscopically remove one or more osteochondral plugs from the defect area. A small amount of biological glue is inserted into the defect and the plug is inserted into the surgically created cylindrical defect. The plug is then positioned so that it is flush with the surface of the surrounding hyaline cartilage area and the gap between the side wall of the plug and the wall defining the bore is filled with a fibrinogen thrombin glue having milled cartilage pieces mixed therein. Additives may be applied to the assembly in order to increase chondrocyte migration and proliferation. Stem cells or chondrocytes may also be applied to the construct to restore the matrix. Each allograft construct can support the addition of a variety of chondrogenic stimulating factors including, but not limited to growth factors (FGF-2, FGF-5, FGF-9, IGF-1, TGF-β, BMP-2, BMP-7, PDGF, VEGF), human allogenic or autologous chondrocytes, human allogenic or autologous bone marrow cells, demineralized bone matrix, insulin, insulin-like growth factor-1, transforming growth factor-B, interleukin-1 receptor antagonist, hepatocyte growth factor, platelet-derived growth factor, Indian hedgehog and parathyroid hormone-related peptide or bioactive glue.

The implant is placed in a bore or hole cut in the patient to remove the lesion area and the milled cartilage glue is used to fill the space or gap not occupied by the plug.

It is an object of the invention to provide an allograft implant for joints which provide pain relief, restores normal function and will postpone or alleviate the need for prosthetic replacement.

It is also an object of the invention to provide a cartilage repair implant which is easily placed by the surgeon using an arthroscopic, minimally invasive technique.

It is further an object of the invention to provide an allograft implant procedure which is applicable for both partial and full thickness lesions.

It is still another object of the invention to keep the cartilage particles in place in the defect.

It is an additional object of the invention to provide implant designs and glue formulations that satisfy surgical requirements and are made from available allograft tissue, some of which would otherwise be considered waste and thrown away.

These and other objects, advantages, and novel features of the present invention will become apparent when considered with the teachings contained in the detailed disclosure along with the accompanying drawings.

### BRIEF DESCRIPTION OF THE DRAWINGS

FIG. 1 shows the anatomy of a knee joint;

FIG. 2 shows a schematic mosaicplasty as known in the prior art;

FIG. 3 shows a cross sectional view of the graft cut and a schematic exploded cross sectional view of a cylindrical allograft osteochondral plug assembly with glue in a defect site;

FIG. 4 shows a perspective view of the osteochondral plug used in FIG. 3 with a cartilage fibrin paste in a defect site; and

FIG. 5 shows a perspective view of the osteochondral plug of FIGS. 3 and 4.

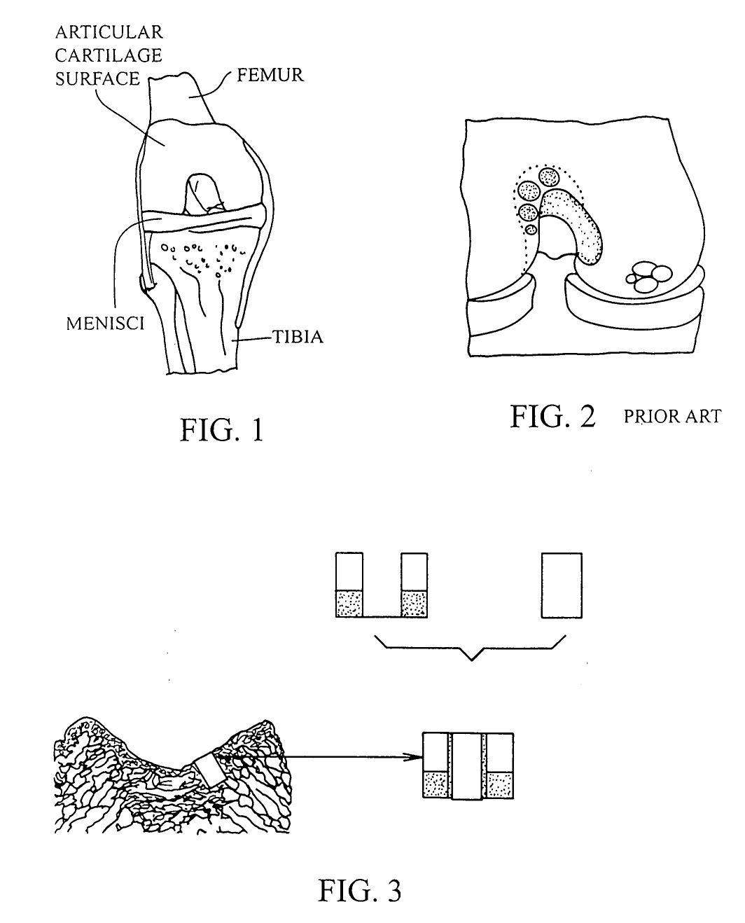

### DESCRIPTION OF THE INVENTION

The terms “tissue” is used in the general sense herein to mean any transplantable or implantable tissue, the survivability of which is improved by the methods described herein upon implantation. In particular, the overall durability and longevity of the implant are improved, and host-immune system mediated responses are substantially eliminated.

The terms “transplant” and “implant” are used interchangably to refer to tissue, material or cells (xenogeneic or allogeneic) which may be introduced into the body of a patient to replace or supplement the structure or function of the endogenous tissue.

The terms “autologous” and “autograft” refer to tissue or cells which originate with or are derived from the recipient, whereas the terms “allogeneic” and “allograft” refer to cells and tissue which originate with or are derived from a donor of the same species as the recipient. The terms “xenogeneic” and “xenograft” refer to cells or tissue which originates with or is derived from a species other than that of the recipient.

The term “glue” refers to a formable mixture of minced or milled pretreated allograft cartilage in a biocomposite carrier.

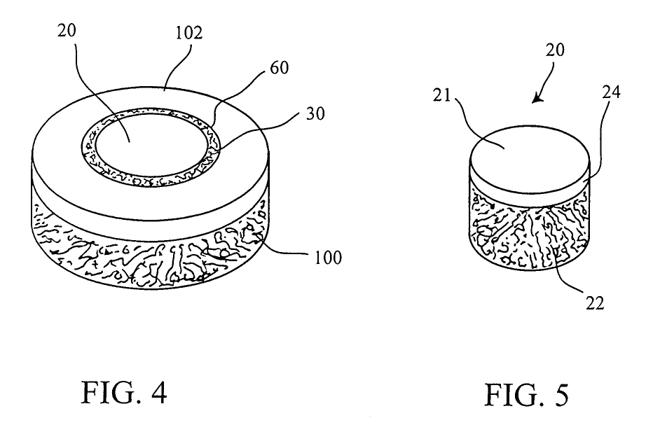

The present invention is directed towards cartilage repair using an osteochondral plug assembly and method of treatment. The preferred embodiment and best mode of the invention is shown in FIGS. 3 to 5. In the production of the invention, an allograft plug 20 having a subchondral bone body 22 and an overlying cap 24 of hyaline cartilage is treated to remove cellular material, chondrocytes and pluripotent mesenchymal cells and proteoglycans and as frozen within the rage of −20° C. to −100° C., preferably −70° C., and lyophilized reducing its water content within the range of about 0.1% to about 8.0%. The cartilage is frozen with liquid nitrogen and ground into particles.

In the treatment for cell and proteoglycan extraction the plug 20 was soaked in hyaluronidase (type IV-s, 3 mg/mL), trypsin (0.25% in monodibasic buffer 3 ml) and the samples were placed in a test tube from 2-18 hours at 37° C. with agitation. It was found that sonication is not a necessary requirement and the times of soaking vary with concentration of hyaluronidase and trypsin and can be as little as 2 hours. The above method of soaking has been previously used on human tissue and is set forth in the Journal of Rheumatology, 12:4, 1985 by Gust Verbruggen et al, entitled “Repair Function in Organ Cultured Human Cartilage Replacement of Enzymatically Removed Proteoglycans During Long term Organ Culture”. After repeated washes with sterile DI water, the hydrated plug samples and cartilage were frozen at −70° C. and lyophilized to reduce water content within a range of about 0.1% to about 8.0%. In an alternative usage, the plug samples and cartilage were frozen after processing.

The osteochondral plug 20 which has been treated as noted above is placed in a blind bore or core 60 which has been cut in the lesion area of the bone 100 of a patient with the upper surface of the cartilage cap 24 being proud or substantially flush with the surface of the cartilage 102 remaining at the area being treated. The length of the osteochondral plug 20 is preferably the same as the depth of the bore 60 so that the base of the plug implant is supported by the bone base 61 of the bore and the articular cartilage cap 24 is level with the articular cartilage 102. With such load bearing support the graft surface is not damaged by excess weight or bearing loads known to cause micromotion interfering with the graft interface producing fibrous tissue interfaces and subchondral cysts.

The plug 20 is movable within bore 60 while resting on the base 61 of the bore 60 and if centered in the bore 60 does not touch the side walls 62 of the bore forming a gap 64 or if touching does not have an interference fit. The distance or gap 64 from the plug 20 to the side wall 62 is preferably less than 2 mm and most preferably from 10 to 1000 microns from the side wall 62. The osteochondral plug 20 which is referred to as a plug is envisioned in various shapes namely, a cylindrical shape and a scalloped shape.

The remainder of the implant area is filled with a milled or minced cartilage mixture 30 having a size generally less than 1 mm together with a fibrin glue as will be more fully described and one or more of the following additives. The additives are one or more of chondrogenic stimulating factors including, but not limited to growth factors (FGF-2, FGF-5, FGF-9, IGF-1, TGF-β, BMP-2, BMP-7, PDGF, VEGF), human allogenic or autologous chondrocytes, human allogenic cells, human allogenic or autologous bone marrow cells, human autologous and allogenic human stem cells, demineralized bone matrix, insulin, insulin-like growth factor-1, interleukin-1 receptor antagonist, hepatocyte growth factor, platelet-derived growth factor, Indian hedgehog and parathyroid hormone-related peptide. The milled cartilage has been lyophilized so that its water content ranges from 0.01% to 8.0% and with the cartilage ranging from 5.0% to 35% by weight.

If desired demineralized or partially demineralized bone powder having a size range from 200 to 850 microns with a weight ranging from 1% to 35% of the cartilage mixture can be added to the milled cartilage glue mixture 30. Either autologous or allogeneic cells can be deposited into the defect area but preferably allogeneic cells such as chondrocytes are added in a range of 10 million to 100 million cells per cc of mixture and more preferably 20 to 40 million cells or may be deposited directly onto the defect area prior to insertion of the plug or after the plug has been deposited.

Suitable organic glue material as described below can be used to keep the implant fixed in place (centered) or positioned as desired in the implant area.

A non-viable or decellularized osteochondral plug consisting of a subchondral bone base and overlying cartilage cap is treated with a solution or variety of solutions to remove the cellular debris as well as the proteoglycans as noted in the treatment described above. It is believed that this removal provides signaling to stimulate the surrounding chondrocytes and also the host's bone marrow and other mesenchymal stem cells to migrate into the graft to proliferate and form new proteoglycans and other factors producing new matrix. The diameter or diagonal of the plug ranges from 1 mm to 30 mm but is preferably 3 mm to 10 mm which is small enough to fit through the endoscopic cannula, but large enough to minimize the number of plugs needed to fill large defects. Since the plug does not engage the sides of the ring and floats in ring area it is important that the gap between the plug and the side wall of the ring cut be less than 2 mm and preferably ranging from 10 microns to 1000 microns. This size provides good results at the recipient site and provides a more confluent hyaline surface. The thickness of subchondral bone can be modified to match the anatomy of the patient so that the surface cartilage of the plug will be even with and follow the surface cartilage of the host tissue. The treated plug also creates a more porous matrix, which allows more cells to enter. The plug and minced hyaline cartilage can be stored frozen or freeze dried and support any of the mentioned chondrogenic stimulating factors. The plug can be inserted arthroscopically similar to the mosaicplasty procedure or through an open incision. The plug can be made in various dimensions depending on the size of the defect being treated.

A fibrin glue 30 which is mixed with milled or minced cartilage is injected into the defect after the plug or plugs are inserted or can be injected before insertion of the plug(s). The fibrin glue fills the gap or space 64 between the plug and the bore wall. Thus, the plug or plugs initially are moveable in the defect bore area until the polymerization of the fibrin glue. For larger defects requiring more than one plug, the fibrin glue also fills the space between the plugs.

The composite fibrin glue mixed with the milled cartilage is formed with a bovine fibrinogen (e.g., SIGMA F-8630), thrombin (e.g., SIGMA T-4648) and aprotinin (e.g., SIGMA A6012). It is also noted that human derived fibrinogen, thrombin and aprotinin can be used.

In the preferred embodiment and in the Examples noted below 283 mg of fibrinogen were dissolved in 2.5 ml of calcium free phosphate buffered saline and 14 mg thrombin was dissolved in 100 μL of sterile water to form a 1:20 dilution. 1 μL aprotinin (15,000 units) was added into the fibrinogen. Milled cartilage particles having a size ranging from 0.01 mm to 1.0 mm were added to either the fibrinogen or thrombin prior to mixing the two together.

### EXAMPLE 1

Allograft cartilage particles having a size ranging from 0.01 mm to 0.21 mm were added to and mixed with fibrinogen solution and 30 μL of fibrinogen solution was inserted in a first automated pipette. The pipette tip was changed, the pipette was set to 60 μL and 30 μL of thrombin solution was taken into the pipette resulting in a mixed solution. The mixed solution was delivered immediately over and into the gap between the bore side wall and the plug and the fibrin glue was allowed to polymerize for 3 minutes at room temperature.

### EXAMPLE 2

Allograft cartilage particles having a size ranging from 0.01 mm to 0.21 mm were added to and mixed with thrombin and 30 μL of thrombin solution was inserted in a automated pipette. The pipette tip was changed and 30 μL of fibrinogen solution was taken into the pipette resulting in a mixed solution. The mixed solution was delivered immediately into and over the gap between the bore side wall and the plug and the fibrin glue was allowed to polymerize for 3 minutes at room temperature.

The operation of placing a preshaped allograft implant assembly in a cartilage defect, utilizes a subchondral bone and an overlying cartilage cap plug which has been treated to remove cellular debris and proteoglycans and milled cartilage in a carrier. The steps of the operation are: (a) drilling a hole which can be in the form of a cylindrical bore in a patient at a site of a cartilage defect to a depth which is equal to the length of the bone and cartilage cap plug implant, (b) placing a preshaped osteochondral plug having a cross section which is less than the cross sectional area of the cylindrical bore leaving a gap between the side wall of the plug and the side wall of the bore ranging between 10 microns and 2000 microns, preferably between 100 microns and 1000 microns, with a length which is equal to or slightly greater than the depth of the bore allowing the structure to be moveable within the bore and (c) placing a mixture of milled cartilage in a fibrinogen thrombin solution in the gap area around the preshaped osteochondral plug and allowing the same to polymerize and (d) adding a solution of fibrinogen or thrombin over the polymerized mixture.

The principles, preferred embodiments and modes of operation of the present invention have been described in the foregoing specification. However, the invention should not be construed as limited to the particular embodiments which have been described above. Instead, the embodiments described here should be regarded as illustrative rather than restrictive. Variations and changes may be made by others without departing from the scope of the present invention as defined by the following claims:

What we claim is:

1. A method of placing a preshaped allograft implant assembly in a cartilage defect, said assembly comprising a subchondral bone and an overlying cartilage cap plug which has been treated to remove cellular debris and proteoglycans and minced cartilage in a carrier comprising the steps of:
(a) drilling a cylindrical hole in a patient at a site of a cartilage defect to a depth which equal to or less than the length of the bone and cartilage cap plug implant to be placed therein forming a blind bore;
(b) placing a preshaped osteochondral plug having a cross section which is less than the cross sectional area of the bore with a gap between the exterior surface of the plug and at least one side wall defining the drilled bore being less than 2 mm allowing the implant to be laterally moveable within said bore in the cylindrical hole;
(c) mixing minced allograft cartilage in a fibrinogen thrombin solution; and
(d) placing the minced cartilage in fibrinogen thrombin solution in the gap between the plug and at least one side wall defining the bore and allowing the cartilage and solution to polymerize.

2. A method as claimed in claim 1 including an additional step of adding a thrombin solution over the polymerized mixture.

3. A method as claimed in claim 1 including an additional step of adding a fibrinogen solution over the polymerized mixture

4. A method as claimed in claim 1 wherein said gap ranges from between 10 microns and 1000 microns.

5. A method as claimed in claim 1 wherein said minced cartilage ranges from about 0.01 mm to about 0.12 mm in size.

6. A method as claimed in claim 1 wherein said assembly includes adding chondrocyte cells from one or more of a group consisting of allograft and autograft.

7. A method as claimed in claim 6 wherein said chondrocyte cells are added in amount ranging from 10.0×10⁶ to 10.0×10⁷.

8. A method as claimed in claim 6 wherein said chondrocyte cells are added in amount ranging from 2.0×10⁷ to 4.0×10⁷.

9. A method as claimed in claim 1 wherein said assembly includes adding a chondrogenic factor taken from a group consisting of growth factors (FGF-2, FGF-5, FGF-9, IGF-1, TGF-β, BMP-2, BMP-7, PDGF, VEGF), human allogenic or autologous chondrocytes, human allogenic cells, human allogenic or autologous bone marrow cells, human autologous and allogenic human stem cells, demineralized bone matrix, insulin, insulin-like growth factor-1, interleukin-1 receptor antagonist, hepatocyte growth factor, platelet-derived growth factor, Indian hedgehog and parathyroid hormone-related peptide.

10. A method of placing a preshaped allograft implant assembly in a cartilage defect, said assembly comprising a subchondral bone and an overlying cartilage cap plug which has been treated to remove cellular debris and proteoglycans and minced cartilage in a carrier comprising the steps of:
(a) drilling a hole in a patient at a site of a cartilage defect to a depth which is equal to or less than the length of a bone and cartilage cap plug implant to be placed therein forming a blind bore;
(b) placing a preshaped osteochondral plug having a cross section which less than the cross sectional area of the bore with a gap ranging from between 10 microns and 1000 microns in size between the exterior surface of the plug and one or more side walls defining the drilled bore being less than 2 mm allowing the implant to be laterally moveable within said bore in the cylindrical hole;
(c) mixing minced allograft cartilage in a fibrinogen thrombin solution; and
(d) placing the minced cartilage in fibrinogen thrombin solution in the gap between the plug and the one or more side walls defining the blind bore and allowing the cartilage and solution to polymerize.

11. A method as claimed in claim 10 wherein said minced cartilage ranges from about 0.01 mm to about 0.12 mm in size.

12. A method as claimed in claim 10 including a separate step of adding a fibrinogen solution over the polymerized solution.

13. A method as claimed in claim 10 wherein said fibrinogen thrombin mixture is of equal quantity.

14. A method as claimed in claim 10 including the step of adding chondrocyte cells from one or more of a group consisting of allograft and autograft chondrocyte cells to said plug.

15. A method as claimed in claim 14 wherein said chondrocyte cells are added in amount ranging from 2.0×10⁷ to 4.0×10⁷.

16. A method as claimed in claim 14 wherein said chondrocyte cells are added in amount ranging from 10.0×10⁶ to 10.0×10⁷.

17. A method as claimed in claim 10 including the step of adding chondrocyte cells from one or more of a group consisting of allograft and autograft chondrocyte cells to said fibrinogen thrombin solution.

18. A method as claimed in claim 17 wherein said chondrocyte cells are added in amount ranging from 2.0×10⁷ to 4.0×10⁷.

19. A method as claimed in claim 17 wherein said chondrocyte cells are added in amount ranging from 10.0×10⁶ to 10.0×10⁷.

20. A method as claimed in claim 10 where said mixed solution is allowed to polymerize for about 3 minutes.

21. A method as claimed in claim 10 including an additional step of adding a thrombin solution over the polymerized mixture.

22. A method as claimed in claim 10 including an additional step of adding a fibrinogen solution over the polymerized mixture.

23. A method as claimed in claim 10 wherein said assembly includes adding a chondrogenic factor taken from a group consisting of growth factors (FGF-2, FGF-5, FGF-9, IGF-1, TGF-β, BMP-2, BMP-7, PDGF, VEGF), human allogenic or autologous chondrocytes, human allogenic cells, human allogenic or autologous bone marrow cells, human autologous and allogenic human stem cells, demineralized bone matrix, insulin, insulin-like growth factor-1, interleukin-1 receptor antagonist, hepatocyte growth factor, platelet-derived growth factor, Indian hedgehog and parathyroid hormone-related peptide.
